(19) United States
(12) Patent Application Publication
(10) Pub. No.: US 2026/0076942 A1
(43) Pub. Date: Mar. 19, 2026

(54) ARYL-IMINO TRYPTOPHAN DERIVATIVES

(71) Applicant: Renovel Innovations, Inc., Fremont, CA (US)

(72) Inventors: Bishwajit NAG, Union City, CA (US); Ananda SEN, Castro Valley, CA (US); Nitish NAG, Union City, CA (US); Srinivasan NARASIMHAN, Chennai (IN); Rupali AGARWAL, Fremont, CA (US)

(73) Assignee: Renovel Innovations, Inc., Fremont, CA (US)

(21) Appl. No.: 19/330,918

(22) Filed: Sep. 17, 2025

Related U.S. Application Data
(60) Provisional application No. 63/695,720, filed on Sep. 17, 2024.

(51) Int. Cl. A61K 31/405; A61P 35/00; C07D 209/20

(52) U.S. Cl. CPC A61K 31/405; A61P 35/00; C07D 209/20

(57) ABSTRACT

Novel Aryl-imino tryptophan derivatives showing anti-cancer activity.

### PRIORITY

The application takes priority from and claims the benefit of 63/695,720, filed 17 Sep. 2024, incorporated by reference herein.

### FIELD OF THE INVENTION

Aryl-imino tryptophan derivatives have potential applications for enzyme inhibition of overexpressed cancer cells to induction of apoptosis in various cancer cell lines. Induction of apoptosis in cancer cells involves triggering the natural process of programmed cell death to eliminate cancerous cells. This is a key strategy in the development of effective cancer therapies.

### BACKGROUND OF THE INVENTION

Cancer is a disease in which the cells of the body grow uncontrollably. The rapid reproduction of abnormal cells can spread to surrounding healthy tissues and organs. The cause and progress of cancer is influenced by a combination of genetic, environmental, and lifestyle factors such as poor diet, inadequate fruit and vegetable intake, tobacco use, alcohol consumption, high body mass index, lack of physical activity, exposure to carcinogens and infections like human papillomavirus (HPV) and hepatitis.

Cancer affects nearly 20 million people worldwide each year. The mortality rate from cancer is also high with nearly 10 million deaths per year. The most common cancers are lung, breast, colorectal, and prostate cancers. Most cancers can be prevented by maintaining healthy weight, exercising regularly, eating a healthy diet, limiting alcohol consumption, not smoking, avoiding sun exposure, preventing infections, and getting screened regularly. These lifestyle changes can also go a long way toward preventing other serious chronic diseases, like heart disease, stroke, diabetes, and osteoporosis.

### BRIEF DESCRIPTION OF THE INVENTION

The invention comprises a series of novel Aryl-imino tryptophan derivatives showing anti-cancer activity.

### SUMMARY OF THE DRAWINGS

FIG. 1: Shows a box-whisker plot for each compound with cell lines grouped by tissue type and agent activity is represented by Response Area.

FIG. 2: Graphs showing data for various cancer lines.

FIG. 3: Graphs showing IC₅₀ of all the cancer cell lines used in the study with RNV-3006.

FIG. 4: Graphs showing IC₅₀ of all the cancer cell lines used in the study with RNV-3007

FIG. 5: Graphs showing IC₅₀ of all the cancer cell lines used in the study with RNV-3053.

FIG. 6: Data showing body weight for RNV-3001

FIG. 7: Data for RNV-3001

FIG. 8: Clinical score data for RNV-3001

FIG. 9: Activity score data for RNV-3001

FIG. 10: Microscopy data for RNV-3001

FIG. 11: Table showing 6 synthetic structures of the invention.

### DETAILED DESCRIPTION OF THE INVENTION

A series of Aryl-imino tryptophan derivatives compounds were screened for anti-cancer activity in in-vitro cell-based and in-vivo study in mice. The compounds are shown in the claims.

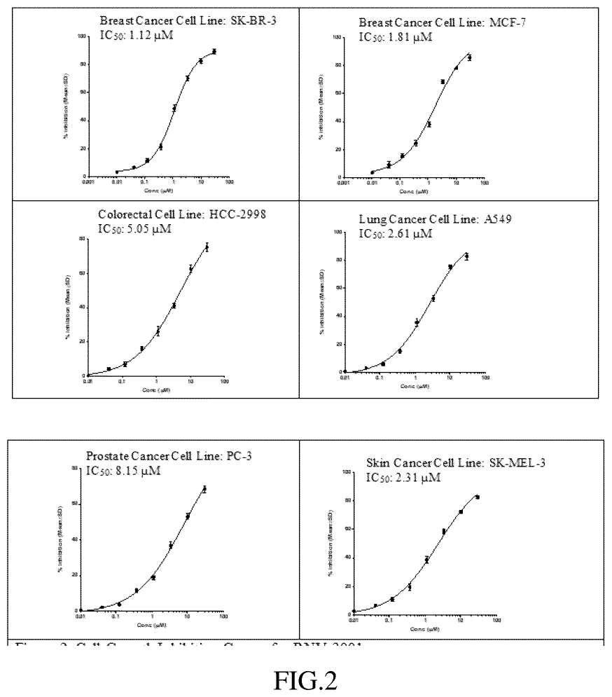

The present invention relates to a series of novel aryl-imino tryptophan derivatives that exhibit significant anti-cancer activity in both in-vitro and in-vivo models. These derivatives are structurally based on indole-containing tryptophan analogs wherein the imino substituent is conjugated to various aryl or substituted aryl groups. Exemplary compounds include RNV-3001, RNV-3006, RNV-3007, RNV-3008, RNV-3053, and RNV-3054, each differing in the nature of the aryl substituent as seen in FIG. 2. The compounds were synthesized through condensation reactions of indole-tryptophan derivatives with aldehydes or acylating agents, and isolated as stable crystalline materials. These derivatives form pharmaceutically acceptable salts, solvates, tautomers, stereoisomers, and polymorphs, thereby broadening their therapeutic applicability.

In vitro studies demonstrated that these compounds inhibit proliferation of diverse human cancer cell lines, including breast (MCF-7, SK-BR-3), colorectal (HCC-2998, COLO-320), lung (A549, NCI-H460), prostate (DU-145, PC-3, LNCaP), and skin (A-375, SK-MEL-3) cancers. Potent activity was observed, for example, with RNV-3001 yielding IC₅₀ values of 1.12 μM in SK-BR-3 and 2.61 μM in A549, while RNV-3006 achieved IC₅₀ values of 0.86 μM in MCF-7 and 2.54 μM in SK-BR-3. RNV-3053 demonstrated sub-micromolar potency against NCI-H460 lung cancer (0.96 μM) and A-375 melanoma (1.72 μM). These values compare favorably with reference chemotherapeutics, supporting their development as targeted anti-cancer agents.

In vivo efficacy was confirmed in a DMBA-induced breast cancer mouse model, wherein treatment with RNV-3001 at 50 mg/kg significantly reduced tumor burden, decreased clinical scores, increased physical activity, and restored normal histological architecture in mammary tissue. Body weight gain associated with tumor burden was reversed, with a 27% reduction relative to untreated controls. Collectively, these findings support the therapeutic potential of aryl-imino tryptophan derivatives as anti-cancer agents capable of inducing apoptosis, reducing tumor progression, and improving clinical outcomes.

A number of embodiments are described in the claims, and include the following.

An aryl-imino tryptophan derivative of formula I, or a pharmaceutically acceptable salt, solvate, tautomer, stereoisomer, or polymorph thereof, wherein the imino substituent is conjugated to an aryl or substituted aryl group.

The compound as described herein, wherein the aryl substituent is selected from hydroxynaphthyl, methoxyphenyl, chlorophenyl, nitrophenyl, hydroxyphenyl, or cinnamoyl.

A pharmaceutical composition comprising at least one compound as described herein and a pharmaceutically acceptable excipient.

A method of treating cancer comprising administering to a subject in need thereof an effective amount of a compound as described herein.

The method of claim 4, wherein the cancer is selected from breast, colorectal, lung, prostate, or skin cancer.

Below are versions of the claimed invention incorporating quantities clinical aspects. They are just examples and are not meant to limit the invention in any way.

The compound as described herein, wherein administration of RNV-3001 results in inhibition of SK-BR-3 breast cancer cells with an IC₅₀ of about 1.12 μM and A549 lung cancer cells with an IC₅₀ of about 2.61 μM.

The compound as described herein, wherein administration of RNV-3006 inhibits MCF-7 breast cancer cells with an IC₅₀ of about 0.86 μM and HCT-116 colorectal cancer cells with an IC₅₀ of about 5.85 μM.

The compound as described herein, wherein administration of RNV-3007 inhibits DU-145 prostate cancer cells with an IC₅₀ of about 0.87 μM.

The compound as described herein, wherein administration of RNV-3053 inhibits NCI-H460 lung cancer cells with an IC₅₀ of about 0.96 μM and A-375 melanoma cells with an IC₅₀ of about 1.72 μM.

The method of claim 4, wherein treatment with RNV-3001 at 50 mg/kg in a DMBA-induced breast cancer mouse model reduces the number of tumors per mouse and decreases clinical scores with statistical significance (P<0.0001).

Exemplary embodiments are provided on the following pages. The text and figures are provided solely as examples to aid the reader in understanding the invention. They are not intended and are not to be construed as limiting the scope of this invention in any manner. Although certain embodiments and examples have been provided, it will be apparent to those skilled in the art based on the disclosures herein that changes in the embodiments and examples shown may be made without departing from the scope of this invention.

A series of Aryl-imino tryptophan derivatives compounds were screened for anti-cancer activity in in-vitro cell-based and in-vivo study in mice.

In a cell-based study, RNV-3001 showed a decrease in human brain and pancreatic cancer cell growth and RNV-3007 showed a decrease in the human pancreatic cancer cell growth. In a subsequent cell-based study, the compounds RNV-3001, RNV-3006, RNV-3007, RNV-3008, RNV-3053, and RNV-3054 were subjected to anti-cancer activity in breast, colorectal, lung, prostate, and skin cancer cell lines. RNV-3007 showed a GI₅₀ above the median value for breast, lung, prostate, and skin cancer cell lines. In another subsequent cell-based screening study, RNV-3001 inhibited cell growth in breast, colorectal, lung and skin cancers, RNV-3006 inhibited cell growth in breast, colorectal, lung and skin cancers, RNV-3007 inhibited the growth of breast, prostate and lung cancers, and RNV-3053 inhibited the growth of colorectal, lung, prostate and skin cancer.

In a 90-day DMBA-induced breast cancer study, treatment with RNV-3001 decreased the DBMA-induced increase of number of breast tumors per mice and decreased DMBA-induced increase of clinical scores. RNV-3001 also showed an increase in physical activity. The histological evaluation on the breast tissues showed that treatment with RNV-3001 increases the ratio of pink cytoplasm relative to the purple cell nucleus in the duct, compared with the DMBA control group of mice. From these studies, it is concluded that Aryl-imino tryptophan derivative compounds have the potential to be developed further for anti-cancer activities.

The present invention relates to Aryl-imino tryptophan compound 1

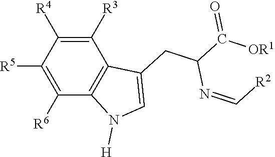

wherein each of R1, R3, R4, R5 and R6 is a Hydrogen; and
R2 is selected from the group consisting of:

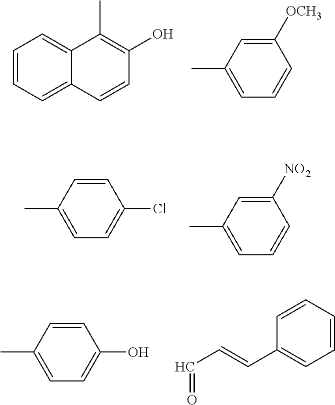

and pharmaceutically acceptable salts thereof.

and its derivatives represented by formula 1, their analogs, their tautomeric forms, their stereoisomers, their polymorphs, their pharmaceutically acceptable salts, their pharmaceutically acceptable solvates, wherein R represents substituents on imino methyl carbon. The present invention also relates to a process for the preparation of the above said novel compounds, their analogs, their tautomeric forms, their stereoisomers, their polymorphs, their pharmaceutically acceptable salts, their pharmaceutically acceptable solvates, novel intermediates and pharmaceutical composites containing them. Tautomeric forms are isomeric forms which exists in a state of equilibrium capable of reacting according to either form. Stereoisomers include configurational isomers, such as cis- and trans double bonds, as well as optically active isomers having different spatial arrangements of their atoms. Polymorphs are molecules which can crystallize in two or more forms. Solvates are molecular or ionic complexes of molecules or ions of solvent with those of a solute. Analogs also include atoms of the same family of the Periodic Table, such as F, Cl, Br and I. Derivatives include compounds resulting from routine functionalizing of atoms, such as, derivatives found by protecting by carboxylation or esterification, respectively. In an embodiment of the present invention, the tryptophan ring represented as ring A can contain one or multiple side chain substituents ranging from hydrogen, phenoxy, amino, sulphonyl, substituted, unsubstituted, straight chain or branched alkyls derivatives, halogens and the like. This also defines the range of substituents accommodated by R3, R4, R5 and R6. In an embodiment of the present invention, the groups represented by R1 on R1O—C═O can be selected from Hydrogen and branched or unbranched alkyl derivatives. In an embodiment of the present invention, the groups represented by R3, R4, R5 and R6 on ring A can be selected from Hydrogen or Halogens like Fluorine, Chlorine, Bromine and Iodine. Pharmaceutically acceptable salts forming part of this invention include base addition salts such as alkali metal salts like Li, Na, and K salts, alkaline earth metal salts like Ca and Mg salts, salts of organic bases such as lysine, arginine, guanidine, diethanolamine, chlorine and the like, ammonium or substituted ammonium salts. Salts may include acid addition salts which are sulphates, nitrates, phosphates, perchlorates, borates, hydrohalides, acetates, tartarates, maleates, citrates, succinates, palmoates, methanesulphonates, benzoates, ascorbates, glycerophosphates, ketoglutarates and the like. Pharmaceutically acceptable solvates may be hydrates or comprising other solvents of crystallization such as alcohols.

More preferably, the present innovation relates to novel Aryl-imino tryptophan compounds. In an embodiment of the present invention, the groups represented as R on N═CH—

The six synthesized compounds of the invention are shown in the table below with IUPAC names.

| Serial.No | RNV | Structure, R² = | IUPAC NAME |
|---|---|---|---|
| 1 | RNV-3001 | 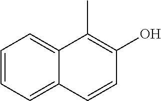 | (Z)-2-((E)-((2-hydroxynaphthalen-1- yl)methylene)amino)-3-(1H-indol-3-yl) acrylic acid |
| 2 | RNV-3006 | 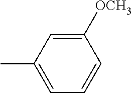 | (E)-3-(1H-indol-3-yl)-2-((3- methoxybenzylidene) amino)propanoic acid |
| 3 | RNV-3007 | 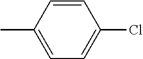 | (E)-2-((4-chlorobenzylidene)amino)- 3-(1H-indol-3-yl)propanoic acid |
| 4 | RNV-3008 | 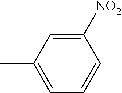 | (Z)-3-(1H-indol-3-yl)-2-((E)-(3- nitrobenzylidene)amino)acrylic acid |
| 5 | RNV-3053 | 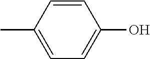 | (E)-2-((4-hydroxybenzylidene)amino)- 3-(1H-indol-3-yl)propanoic acid |
| 6 | RNV-3054 | 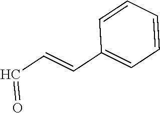 | 2-cinnamamido-3-(1H-indol-3-yl) propanoic acid |

According to another feature of this present invention, few of the analogues synthesized are represented in Table 8. According to another feature of this present invention, there is provided a process for the preparation of the compound represented by the formula I, wherein all symbols are as defined as earlier, as shown in scheme-I.

### Structure, Molecular Weight, IUPAC Name, and Schematic Structure:

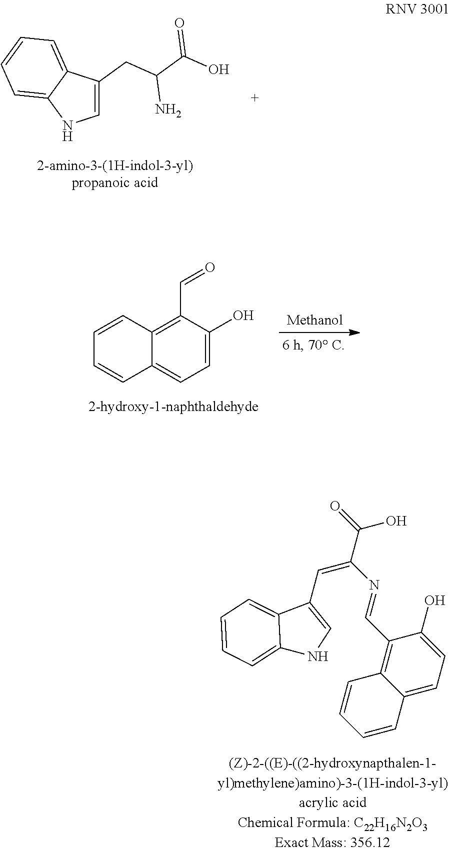 [SMILES: NC(Cc1c[nH]c2ccccc12)C(=O)O ; O=Cc1c(O)ccc2ccccc12 ; O=C(O)C(=C/c1c[nH]c2ccccc12)/N=C/c1c(O)ccc2ccccc12]

### Synthetic Procedure:

Compounds 2-amino-3-(1H-indol-3-yl)-propanoic acid (1 equivalent) and 2-hydroxy-1-naphthaldehyde (1 equivalent) was dissolved in methanol and molecular sieve added and the mixture was stirred for 6 h at 70° C.

Reaction monitored by TLC and after completion of the reaction, the mixture was quenched with water.

The obtained precipitate was filtered and dried at room temperature.

### Structure, Molecular Weight, IUPAC Name, and Schematic Structure:

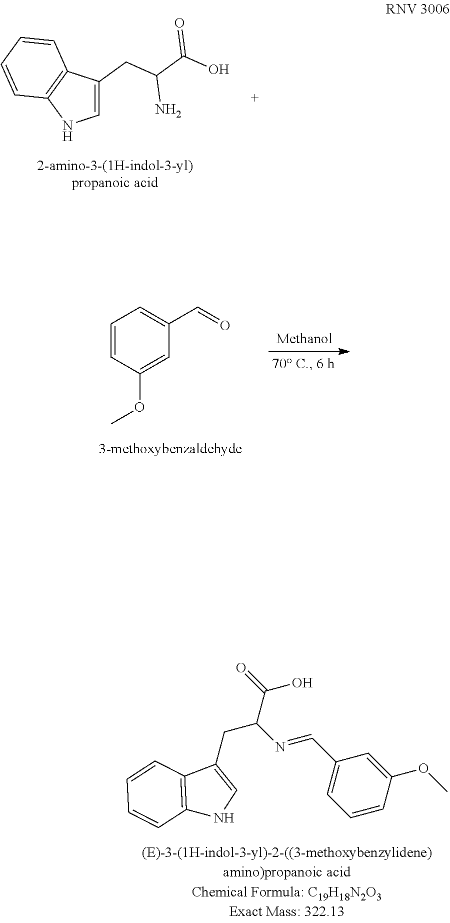

### Synthetic Procedure:

Compounds 2-amino-3-(1H-indol-3-yl)-propanoic acid (1 equivalent) and 3-methoxyaldehyde (1 equivalent) was dissolved in methanol and molecular sieve added and the mixture was stirred for 6 h at 70° C.

Reaction monitored by TLC and after completion of the reaction, the mixture was quenched with water.

The obtained precipitate was filtered and dried at room temperature.

### Structure, Molecular Weight, IUPAC Name, and Schematic Structure:

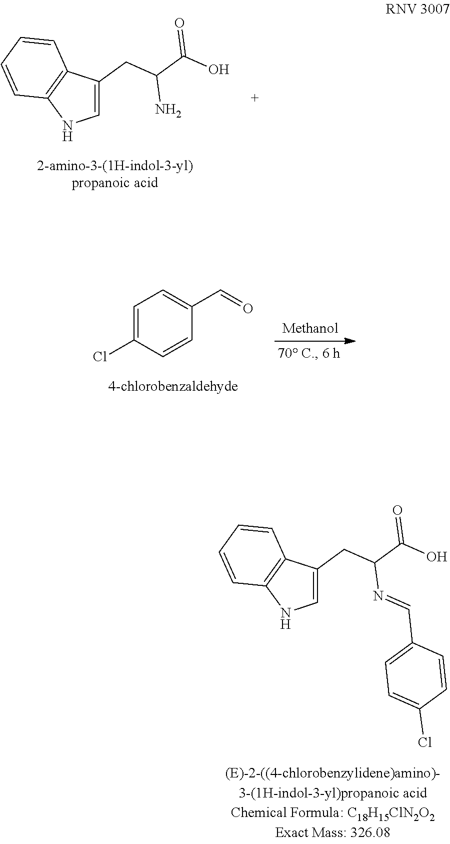 [SMILES: NC(Cc1c[nH]c2ccccc12)C(=O)O ; O=Cc1ccc(Cl)cc1 ; O=C(O)C(Cc1c[nH]c2ccccc12)/N=C/c1ccc(Cl)cc1]

### Synthetic Procedure:

Compounds 2-amino-3-(1H-indol-3-yl)-propanoic acid (1 equivalent) and 4-chloroaldehyde (1 equivalent) was dissolved in methanol and molecular sieve added and the mixture was stirred for 6 h at 70° C.

Reaction monitored by TLC and after completion of the reaction, the mixture was quenched with water.

The obtained precipitate was filtered and dried at room temperature.

### Structure, Molecular Weight, IUPAC Name, and Schematic Structure:

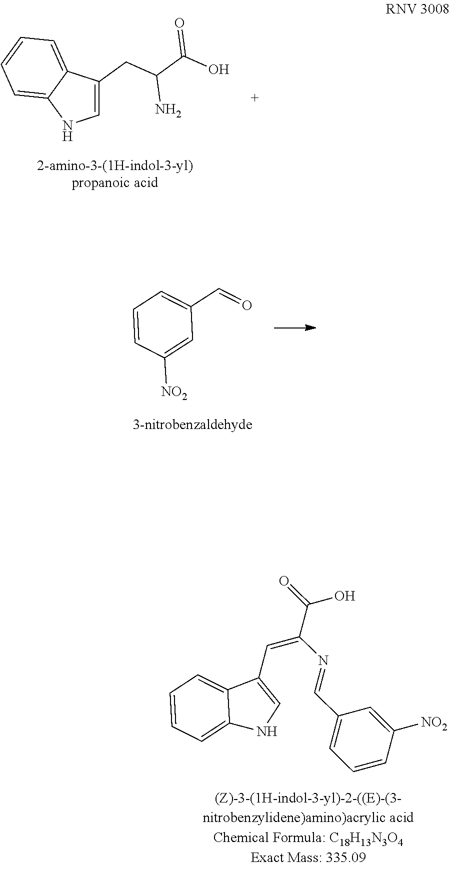 [SMILES: NC(Cc1c[nH]c2ccccc12)C(=O)O ; O=Cc1cccc([N+](=O)[O-])c1 ; O=C(O)C(=C/c1c[nH]c2ccccc12)/N=C/c1cccc([N+](=O)[O-])c1]

### Synthetic Procedure:

Compounds 2-amino-3-(1H-indol-3-yl)-propanoic acid (1 equivalent) and cinnamaldehyde (1 equivalent) was dissolved in methanol and molecular sieve added and the mixture was stirred for 6 h at 70° C.

Reaction monitored by TLC and after completion of the reaction, the mixture was quenched with water.

The obtained precipitate was filtered and dried at room temperature.

### Structure, Molecular Weight, IUPAC Name, and Schematic Structure:

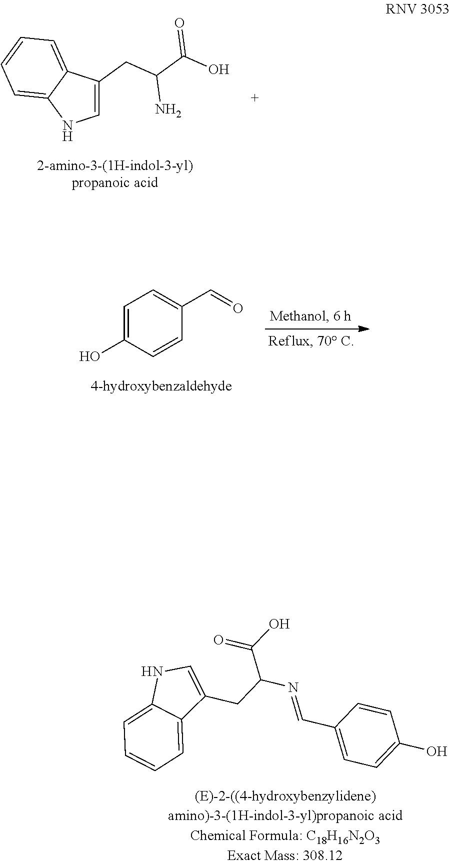 [SMILES: NC(Cc1c[nH]c2ccccc12)C(=O)O ; O=Cc1ccc(O)cc1 ; O=C(O)C(Cc1c[nH]c2ccccc12)/N=C/c1ccc(O)cc1]

### Synthetic Procedure:

Compounds 2-amino-3-(1H-indol-3-yl)-propanoic acid (1 equivalent) and 4-hydroxy benzaldehyde (1 equivalent) was dissolved in methanol and stirred the solution for 6 h at 70° C.

Reaction monitored by TLC and after completion of the reaction, the mixture was quenched with water.

The obtained precipitate was filtered and dried at room temperature.

### Structure, Molecular Weight, IUPAC Name, and Schematic Structure:

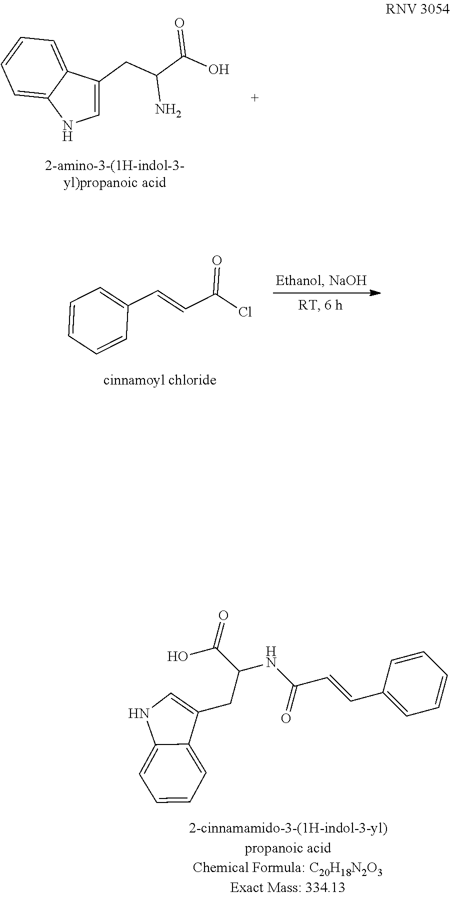 [SMILES: NC(Cc1c[nH]c2ccccc12)C(=O)O ; O=C(Cl)/C=C/c1ccccc1 ; O=C(/C=C/c1ccccc1)NC(Cc1c[nH]c2ccccc12)C(=O)O]

### Synthetic Procedure:

Compounds 2-amino-3-(1H-indol-3-yl)-propanoic acid (1 equivalent) and cinnamoyl chloride (1 equivalent) was dissolved in ethanol.

Add alcoholic NaOH solution to the mixture then kept for room temperature 6 h.

Reaction monitored by TLC and after completion of the reaction, the mixture was quenched with water.

The obtained precipitate was filtered and dried over room temperature.

### In-Vitro Screening 1

U87MG (human brain cancer cell line) and PANC-1 (human pancreatic cancer cell line) were plated in 96-well format (50,000 cells/well) and grown overnight for adherence. Test compounds were added at different concentrations (100, 33, 11, 3.3, 1.1, 0.33, and 0.01 μM, in triplicates) and cells were incubated for 24 hours. At the end of incubation, cell viability was determined by standard MTT method. IC₅₀ was determined using GraphPad Prism. Camptothecin and Cisplatin were used as reference compounds.

TABLE 2
Result of In-Vitro Screening 1

| S. No. | Compound Name | U87MG (IC₅₀ μM) | PANC-1 (IC₅₀ μM) |
|---|---|---|---|
| 1 | RNV-3001 | 21.96 | 7.61 |
| 2 | RNV-3007 | ND | 16.14 |
| 3 | Camptothecin | 13.6 | NA |
| 4 | Cisplatin | NA | 10.53 |

ND: Not Determined;
NA: Not Applicable.
U87MG: Human glioma.
PANC-1: Pancreatic carcinoma

### In-Vitro Screening 2

High throughput screening platform was used to determine single agent activity of compounds across different cell lines. Single agents were dosed with 9 dose points including the no treatment control. The principal assay readout was growth inhibition determined using a 96-hour viability assay. Potency and efficacy metrics were derived from logistic curves fitted to growth inhibition (GI) and inhibition (Inh) data. Growth Inhibition (% GI) was calculated as a measure of cell growth and response to compound treatments.

The compounds were tested on different cancer tissue types with the following cell lines:
Breast (MDA-MB-231, HCC1954, BT-20)
Colorectal (COLO-320, SW480, COLO-205)
Lung (Calu-1, RERF-LC-AI, RERF-LC-MS)
Prostate (22RV1, PC-3, DU-145)
Skin (Hs 936.T, WM-115, HMCB)

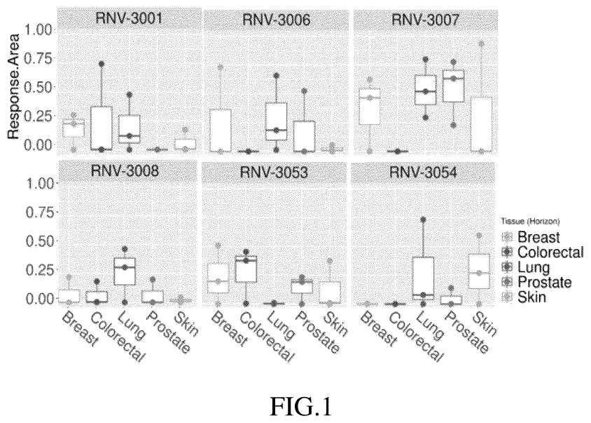

A box whisker plot for each compound was generated. Cell lines are grouped by tissue type (sorted by median response for each tissue type alphabetically, highest response on right), and agent activity is represented by Response Area (area under growth inhibition dose response curve). See FIG. 1. FIG. 1: Box Whisker Plot from In-Vitro Screening 2. The response area against each compound for the five cancer tissues is also shown in Table 3.

TABLE 3
Result of In-Vitro Screening 2

| | Response Area under Growth Inhibition | | | | |
|---|---|---|---|---|---|
| Compound | Breast | Colorectal | Lung | Prostate | Skin |
| RNV-3001 | 0.25 | — | 0.15 | — | — |
| RNV-3006 | — | — | 0.20 | — | — |
| RNV-3007 | 0.45 | — | 0.55 | 0.65 | — |
| RNV-3008 | — | — | 0.35 | — | — |
| RNV-3053 | 0.20 | 0.40 | — | 0.20 | — |
| RNV-3054 | — | — | — | — | 0.25 |

A heat map was also generated for the compounds. Based on the heat map, the following compounds show a measurable activity on different cancer cell lines:
RNV-3001: COLO-320 (Colon Adenocarcinoma—male)
RNV-3006: HCC1954 (Breast cancer)
RNV-3007: PC-3 (Prostatic small cell carcinoma), Hs936. T (Melanoma—male skin cell line), Calu-1 (Lung squamous carcinoma)
RNV-3054: Calu-1 (Lung squamous carcinoma)

### In-Vitro Screening 3

An in-vitro cytotoxicity study of RNV compounds was undertaken against cancer cell lines by MTT assay. The cell viability assay was done in the following cell lines of these cancer types:
Breast (MDA-MB-231, MDA-MB-453, MCF-7, SK-BR-3 & BT-20)
Colorectal (HCT-116, DLD-1, HCC-2998, Colo320 & SW-480)
Lung (A549, NCI-H1975, HOP-62, NCI-H82 & NCI-H460)
Prostate (DU-145, PC3, LNCaP, 22Rv1 & C4-2)
Skin (A-375, A-431, G-361, SK-MEL-28 & SK-MEL-3)

For the cytotoxicity study, 100 μL cell suspension (in complete medium with 10% FBS) was seeded in a 96-well plate (20,000 cells per well), without the test agent and allowed to grow for about 24 hours. After 24 hours of incubation, treatment was carried out, spent media in the wells of 96-well plate was replaced with 200 μL of appropriate concentrations of the test compounds/positive control and incubated for 72 hours at 37° C. in a 5% CO2 atmosphere. After the incubation period, spent media was removed and wells were washed/rinsed with DMEM followed by addition of MTT reagent to a final concentration of 0.5 mg/ml (0.2 μM filter sterilized). The plates were wrapped with aluminum foil to avoid exposure to light, and placed in the incubator for 3 hours. After incubation MTT reagent was removed and 100 μL of DMSO was added. Absorbance was measured on spectrophotometer (Tecan™ Infinite 200Pro) at 570 nm. The percent viability of vehicle treated cells was set to 100% and the % viability of treated cells was estimated relative to the untreated control. The % viability was plotted against the concentration and evaluated for dose response.

Percentage viability was calculated using the following formula: % Viability=100×OD570e/ID570b. Where, OD570e is the mean OD value of the dilutions of test item/positive control; OD570b is the mean OD value of the vehicle control.

### RNV-3001

Table 4 shows the IC₅₀ of all the cancer cell lines used in the study with RNV-3001. Based on the cell-based study results, RNV-3001 showed an IC₅₀ of 1.12 μM in SK-BR-3 and 1.81 μM in MCF-7 (breast cancer), 5.05 μM in HCC-2998 (colorectal cancer), 2.61 μM in A549 (lung cancer), 8.15 μM in PC-3 (prostate cancer) and 2.31 μM in SK-MEL-3 (skin cancer) See FIG. 2.

TABLE 1
RNV-3001 (IC₅₀ in μM)

| Breast Cell Line | MDA-MB-231 | MDA-MB-453 | MCF-7 | SK-BR-3 | BT-20 |
|---|---|---|---|---|---|
| | >30 | 24.99 | 1.81 | 1.12 | 20.14 |
| Colorectal Cell Line | HCT-116 | DLD-1 | HCC-2998 | COLO320 | SW-480 |
| | >30 | 20.77 | 5.05 | 9.46 | 12.78 |
| Lung Cell Line | A549 | NCI-H1975 | HOP-62 | NCI-H82 | NCI-H460 |
| | 2.61 | 13.53 | 4.98 | 4.19 | 10.87 |
| Prostate Cell Line | PC-3 | 22RV1 | C4-2 | DU145 | LNCap |
| | 8.15 | 10.8 | 13.23 | 16.56 | 23.29 |
| Skin Cell Line | A-375 | A-431 | G-361 | SK-MEL-28 | SK-MEL-3 |
| | 4.13 | 3.1 | 8.12 | >30 | 2.31 |

### RNV-3006

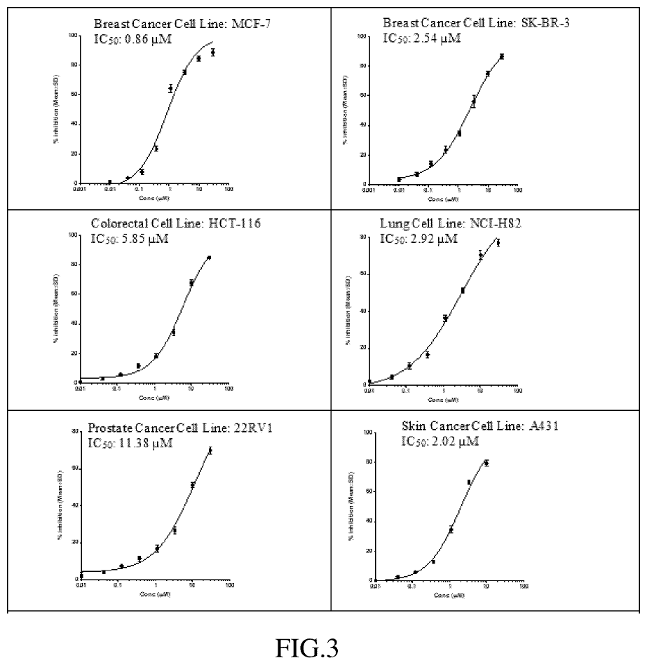

Table 5 shows the IC₅₀ of all the cancer cell lines used in the study with RNV-3006. Based on the cell-based study results, RNV-3006 showed an IC₅₀ of 0.86 μM in MCF-7 and 2.54 μM in SK-BR-3 (breast cancer), 5.85 μM in HCT-116 (colorectal cancer), 2.92 μM in NCI-H82 (lung cancer), 11.38 μM in 22RV1 (prostate cancer) and 2.02 μM in A-431 (skin cancer) See FIG. 3.

TABLE 5
RNV-3006 (IC₅₀ in μM)

| Breast Cell Line | MDA-MB-231 | MDA-MB-453 | MCF-7 | SK-BR-3 | BT-20 |
|---|---|---|---|---|---|
| | 6.83 | 15.96 | 0.86 | 2.54 | >30 |
| Colorectal Cell Line | HCT-116 | DLD-1 | HCC-2998 | COLO320 | SW-480 |
| | 5.85 | 11.99 | 24.39 | 6.31 | 19 |
| Lung Cell Line | A549 | NCI-H1975 | HOP-62 | NCI-H82 | NCI-H460 |
| | 24.07 | 15.71 | 11.18 | 2.92 | 3.24 |
| Prostate Cell Line | PC-3 | 22RV1 | C4-2 | DU145 | LNCap |
| | >30 | 11.38 | 19.23 | 12.3 | >30 |
| Skin Cell Line | A-375 | A-431 | G-361 | SK-MEL-28 | SK-MEL-3 |
| | 10.95 | 2.02 | >30 | >30 | >30 |

### RNV-3007

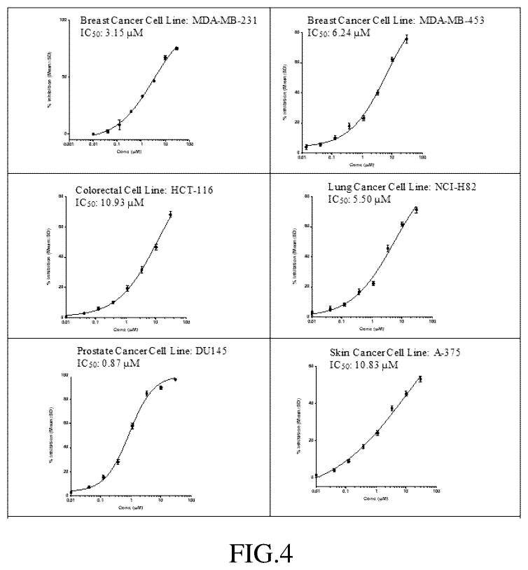

Table 6 shows the IC₅₀ of all the cancer cell lines used in the study with RNV-3007. Based on the cell-based study results, RNV-3007 showed an IC₅₀ of 3.15 μM in MDA-MB-231 and 6.24 μM in MDA-MB-453 (breast cancer), 10.93 μM in HCT-116 (colorectal cancer), 5.5 μM in NCI-H82 (lung cancer) and 0.87 μM in DU145 (prostate cancer) and 10.83 μM in A-375 (skin cancer) (see FIG. 4).

TABLE 6
RNV-3007 (IC₅₀ in μM)

| Breast Cell Line | MDA-MB-231 | MDA-MB-453 | MCF-7 | SK-BR-3 | BT-20 |
|---|---|---|---|---|---|
| | 3.15 | 6.24 | >30 | 14.44 | 15.58 |
| Colorectal Cell Line | HCT-116 | DLD-1 | HCC-2998 | COLO320 | SW-480 |
| | 10.93 | >30 | >30 | >30 | 27.53 |
| Lung Cell Line | A549 | NCI-H1975 | HOP-62 | NCI-H82 | NCI-H460 |
| | >30 | >30 | >30 | 5.5 | >30 |
| Prostate Cell Line | PC-3 | 22RV1 | C4-2 | DU145 | LNCap |
| | >30 | >30 | >30 | 0.87 | 10.73 |
| Skin Cell Line | A-375 | A-431 | G-361 | SK-MEL-28 | SK-MEL-3 |
| | 10.83 | 11.67 | >30 | 22.27 | 11.05 |

### RNV-3053

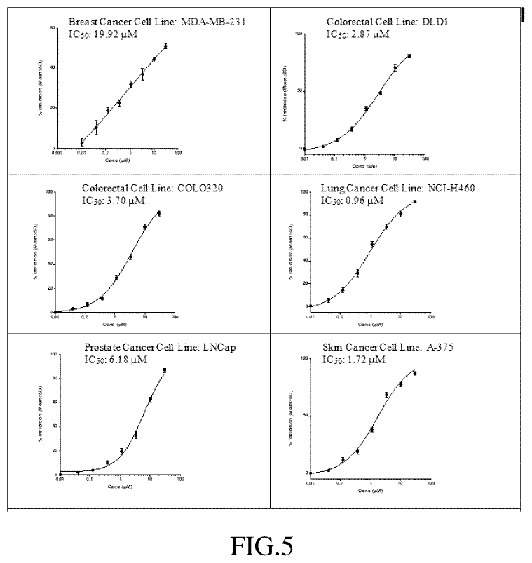

Table 7 shows the IC₅₀ of all the cancer cell lines used in the study with RNV-3053. Based on the cell-based study results, RNV-3053 showed an IC₅₀ of 19.92 M in MDA-MB-231 (breast cancer), 2.87 μM in DLD-1 and 3.70 μM in COLO320 (colorectal cancer), 0.96 μM in NCI-H460 (lung cancer), 6.18 μM in LNCap (prostate cancer) and 1.72 μM in A-375 (skin cancer) See FIG. 5.

TABLE 7
RNV-3053 (IC₅₀ in μM)

| Breast Cell Line | MDA-MB-231 | MDA-MB-453 | MCF-7 | SK-BR-3 | BT-20 |
|---|---|---|---|---|---|
| | 19.92 | >30 | >30 | >30 | >30 |
| Colorectal Cell Line | HCT-116 | DLD-1 | HCC-2998 | COLO320 | SW-480 |
| | 27.41 | 2.87 | >30 | 3.7 | 29.81 |
| Lung Cell Line | A549 | NCI-H1975 | HOP-62 | NCI-H82 | NCI-H460 |
| | 21.82 | >30 | 25.74 | 13.45 | 0.96 |
| Prostate Cell Line | PC-3 | 22RV1 | C4-2 | DU145 | LNCap |
| | 17.79 | 20.69 | 8.54 | >30 | 6.18 |
| Skin Cell Line | A-375 | A-431 | G-361 | SK-MEL-28 | SK-MEL-3 |
| | 1.72 | >30 | 4.3 | 5.43 | 3.62 |

### Animal Study Data

A 90-day study was done to determine the effect of RNV-3001 on DMBA-induced breast cancer model in female Swiss Webster mice. DMBA was administered weekly at 0.5 mg/mouse in 200 μL of sesame oil orally for a total of 3 weeks to all the treatment groups of mice. All the mice were fed with normal pellet diet soaked in corn oil.

Following weekly DMBA treatment, the mice were weighed and randomized into the following treatment and vehicle groups: (1) Group I: Normal control (not administered with DMBA), (2) Group II: DMBA Control, (3) Group III: DMBA+RNV-3001 at 50 mg/kg, (4) Group IV: DMBA+Tamoxifen at 50 mg/kg.

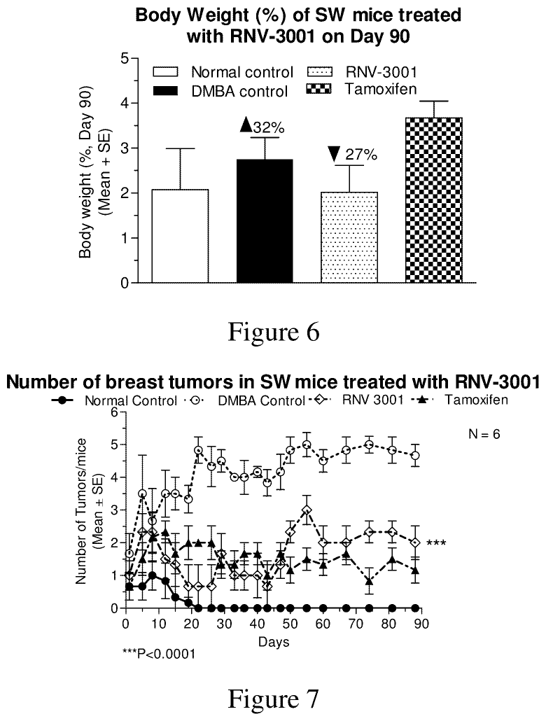

The mice were scored twice a week for (1) Body weight, (2) Number of breast tumors per mice, (3) Clinical score, and (4) Activity score. See FIGS. 6-9.

### Histology Data

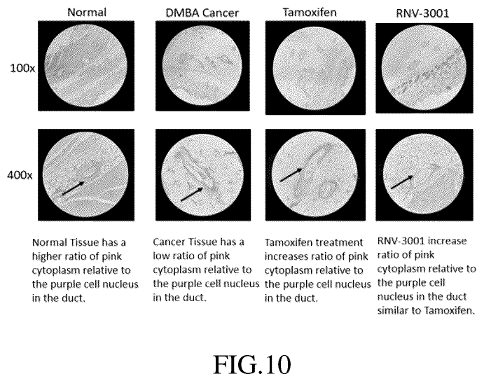

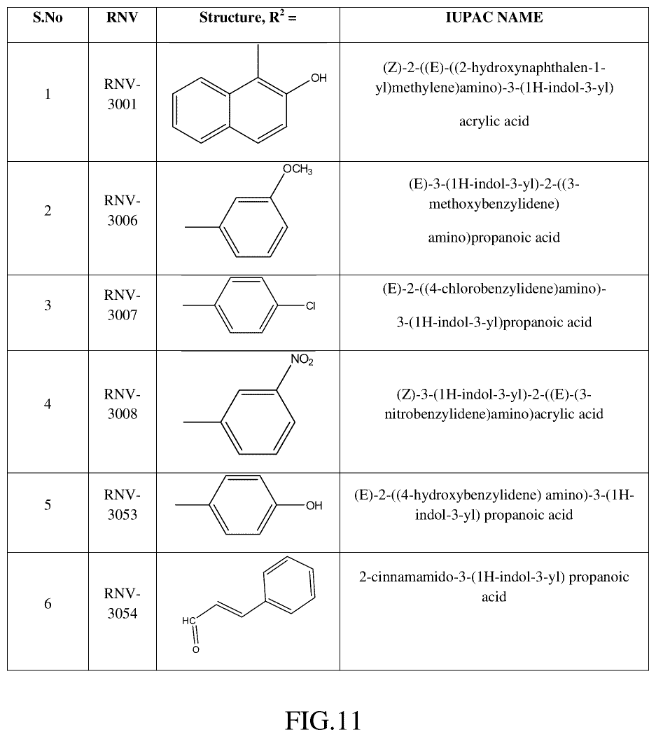

Upon termination, the mice were euthanized by carbon dioxide asphyxiation. The breasts were dissected out and stored in 0.1% formalin for histological evaluation. See FIG. 10

### BRIEF DESCRIPTION OF FIGURES

FIG. 1 shows the box whisker plot for compounds RNV-3001, RNV-3006, RNV-3007, RNV-3008, RNV-3053 and RNV-3054 in different cancer types and shows the growth inhibition dose response curve for each compound.

FIG. 2 shows the cell growth inhibition curves for RNV-3001 with an IC₅₀ of 1.12 μM in SK-BR-3 and 1.81 μM in MCF-7 (breast cancer), 5.05 μM in HCC-2998 (colorectal cancer), 2.61 μM in A549 (lung cancer), 8.15 μM in PC-3 (prostate cancer) and 2.31 μM in SK-MEL-3 (skin cancer).

FIG. 3 shows the cell growth inhibition curves for RNV-3006 with an IC₅₀ of 0.86 μM in MCF-7 and 2.54 μM in SK-BR-3 (breast cancer), 5.85 μM in HCT-116 (colorectal cancer), 2.92 μM in NCI-H82 (lung cancer), 11.38 μM in 22RV1 (prostate cancer) and 2.02 μM in A-431 (skin cancer).

FIG. 4 shows the cell growth inhibition curves for RNV-3007 with an IC₅₀ of 3.15 μM in MDA-MB-231 and 6.24 μM in MDA-MB-453 (breast cancer), 10.93 μM in HCT-116 (colorectal cancer), 5.5 μM in NCI-H82 (lung cancer) and 0.87 μM in DU145 (prostate cancer) and 10.83 μM in A-375 (skin cancer).

FIG. 5 shows the cell growth inhibition curves for RNV-3053 with an IC₅₀ of 19.92 μM in MDA-MB-231 (breast cancer), 2.87 μM in DLD-1 and 3.70 μM in COLO320 (colorectal cancer), 0.96 μM in NCI-H460 (lung cancer), 6.18 μM in LNCap (prostate cancer) and 1.72 μM in A-375 (skin cancer).

FIG. 6 shows the percentage change in body weight in female SW mice treated with RNV-3001. The group of mice treated with RNV-3001 showed a decrease in body weight by 27% compared with the DMBA control group.

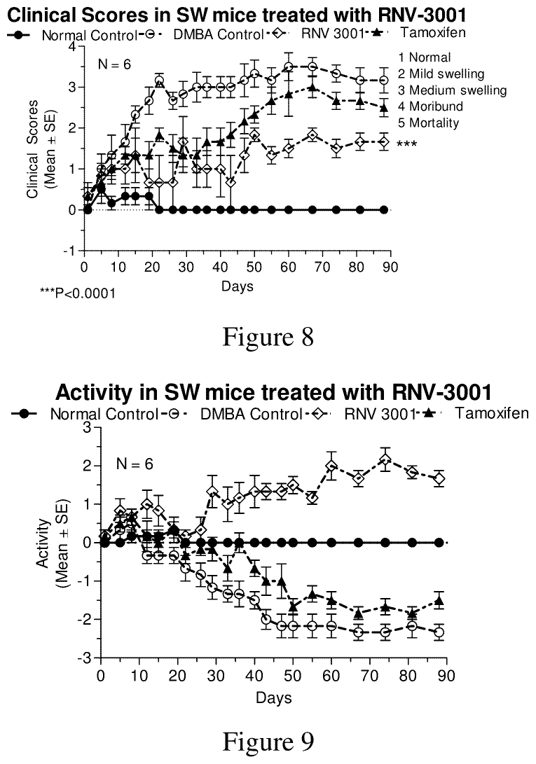

FIG. 7 demonstrates the number of breast tumors in female SW mice treated with RNV-3001. The mice treated with RNV-3001 shows significant reduction in the breast tumors (P<0.0001). FIG. 8 shows the clinical score in female SW mice treated with RNV-3001. DMBA control group shows an increase in clinical scores when compared with the normal group of mice. The group of mice treated with RNV-3001 significantly reversed the clinical score towards the non-treated normal group of mice (P<0.0001).

FIG. 9 shows the score of physical activity in female SW mice treated with RNV-3001. DMBA control group shows a decrease in activity scores when compared with the normal group of mice. The group of mice treated with RNV-3001 significantly increased the activity score (P<0.0001).

### Further Embodiments of the Invention

Screening 1: An initial cell-based screening was undertaken on the compounds RNV-3001 and RNV-3007 in human brain cancer (U87MG) and pancreatic (PANC-1) cell lines. RNV-3001 showed a decrease of human brain cancer cell growth with an IC₅₀ of 21.96 μM. RNV-3001 also showed a decrease of human pancreatic cancer cell growth with an IC₅₀ of 7.61 μM. RNV-3007 showed a decrease of human pancreatic cancer cell growth with an IC₅₀ of 16.14 μM.

Screening 2: A cell-based screening was undertaken with RNV-3001, RNV-3006, RNV-3007, RNV-3008, RNV-3053, and RNV-3054 compounds in breast (MDA-MB-231, HCC1954, BT-20), colorectal (COLO-320, SW480, COLO-205), lung (Calu-1, RERF-LC-AI, RERF-LC-MS), prostate (22RV1, PC-3, DU-145), and skin (Hs 936.T, WM-115, HMCB) cancer cell lines. Based on the whisker plots and response area, RNV-3007 shows GI₅₀ above the median value for breast, lung, prostate, and skin cancer cell lines. RNV-3001, RNV-3006, RNV-3008, RNV-3053, and RNV-3054 showed some efficacy which was below the median GI₅₀ value.

Screening 3: An in-vitro cell screening was conducted with RNV-3001, RNV-3006, RNV-3007, RNV-3008, RNV-3053, and RNV-3054 in breast (MDA-MB-231, MDA-MB-453, MCF-7, SK-BR-3 & BT-20), colorectal (HCT-116, DLD-1, HCC-2998, Colo320 & SW-480), lung (A549, NCI-H1975, HOP-62, NCI-H82 & NCI-H460), prostate (DU-145, PC3, LNCaP, 22Rv1 & C4-2), and skin (A-375, A-431, G-361, SK-MEL-28 & SK-MEL-3) cancer cell lines.

The study results showed that RNV-3001 inhibited the growth of the breast cancer cell lines MCF-7 with an IC₅₀ of 1.81 μM and SK-BR-3 with an IC₅₀ of 1.12 μM. RNV-3001 inhibited the growth of colorectal cancer cell line HCC-2998 with an IC₅₀ of 5.05 μM. RNV-3001 inhibited the growth of lung cancer cell lines A549 with an IC₅₀ of 2.61 μM, HOP-62 with an IC₅₀ of 4.98 μM, NCI-H82 with an IC₅₀ of 4.19 μM. RNV-3001 inhibited the growth of skin cancer cell lines A-375 with an IC₅₀ of 4.13 μM, A-431 with an IC₅₀ of 3.1 μM, SK-MEL-3 with an IC₅₀ of 2.31 μM.

The study results also show that RNV-3006 inhibited the growth of breast cancer cell line MCF-7 with an IC₅₀ of 0.86 μM, SK-BR-3 cell line with an IC₅₀ of 2.54 μM. RNV-3006 inhibited the growth of lung cancer cell line NCI-H82 with an IC₅₀ of 2.92 μM, NCI-H460 cell line with an IC₅₀ of 3.24 μM. RNV-3006 inhibited the growth of colorectal cell line HCT-116 with an IC₅₀ of 5.85 μM and skin cancer cell line A-431 with an IC₅₀ of 2.02 μM.

RNV-3007 inhibited the growth of breast cancer cell lines MDA-MB-231 with an IC₅₀ of 3.15 μM and MDA-MB-453 with an IC₅₀ of 6.24 μM. In the lung cancer cell line NCI-H82, RNV-3007 inhibited its growth with an IC₅₀ of 5.5 μM. RNV-3007 inhibited the growth of prostate cancer cell line DU145 with an IC₅₀ of 0.87 μM.

RNV-3053 inhibited the growth of colorectal cancer cell lines DLD-1 with an IC₅₀ of 2.87 μM and COLO320 with an IC₅₀ of 3.7 μM. In the lung cancer cell line NCI-H460, RNV-3053 inhibited its growth with an IC₅₀ of 0.96 μM. RNV-3053 inhibited the growth of prostate cancer cell line LNCap with an IC₅₀ of 6.18 μM. RNV-3053 inhibited the growth of skin cancer cell lines A-375 with an IC₅₀ of 1.72 μM, G-361 with an IC₅₀ of 4.3 μM, SK-MEL-28 with an IC₅₀ of 5.43 μM, SK-MEL-3 with an IC₅₀ of 3.62 μM.

Animal Study Model: A 90-day DMBA-induced breast cancer study was undertaken in female Swiss Webster mice. DMBA was administered weekly for 3 weeks to all the treatment groups of mice. Body weight, number of breast tumors per mice, clinical scores, and activity scores were measured.

Body Weight: The group of mice administered with DMBA showed a 32% increase in body weight compared with the normal group of mice. Treatment with RNV-3001 at 50 mg/kg, PO showed a 27% decrease in the body weight compared to the DMBA control group of mice. The group of mice treated with Tamoxifen at 50 mg/kg did not show any change in body weight when compared to the DMBA group of mice.

Number of Breast Tumors Per Mice: The group of mice administered with DMBA showed an increase in the number of breast tumors. Treatment with RNV-3001 at 50 mg/kg, PO showed a significant decrease (P<0.0001) in the number of breast tumors compared to the DMBA control group of mice. The group of mice treated with Tamoxifen showed similar response as compared to the group of mice treated with RNV-3001.

Clinical Score: Clinical score is a value that is used to record the strength or severity of a disease or medical phenomenon. In this study, the group of mice administered with DMBA showed an increase in the clinical score compared with the normal group of mice. Treatment with RNV-3001 at 50 mg/kg, PO showed a significant decrease (P<0.0001) in the clinical score compared to the DMBA control group of mice. The group of mice treated with Tamoxifen (P<0.002) reverses the DMBA-induced increase in clinical scores but not as significantly as the RNV-3001 treated group of mice.

Activity Score: Activity score is the value of the mobility of the animal in the study subjected to the disease. In this study, the group of mice administered with DMBA showed a decrease in the activity compared to the normal group of mice. Treatment with RNV-3001 at 50 mg/kg, PO showed a significant increase (P<0.0001) in the activity score as compared to the DMBA control group of mice. The group of mice treated with Tamoxifen (P<0.03) reverses the DMBA-induced decrease in activity scores but not as significantly as the RNV-3001 treated group of mice.

Upon termination of the study, the breast tissues from the mice were dissected out. A histological evaluation was undertaken on the breast tissues. The DMBA-treated breast cancer tissues showed a low ratio of pink cytoplasm relative to the purple cell nucleus in the duct, compared with normal breast tissues. Treatment with RNV-3001 at 50 mg/kg increases the ratio of pink cytoplasm relative to the purple cell nucleus in the duct, compared with the DMBA control group of mice. The group of mice treated with Tamoxifen at 50 mg/kg showed a similar response as RNV-3001.

1. An Aryl-imino tryptophan compound represented by formula 1, 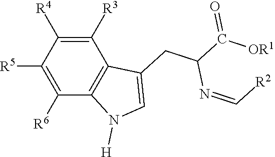
wherein each of R1, R3, R4, R5 and R6 is a Hydrogen; and
R2 is selected from the group consisting of: 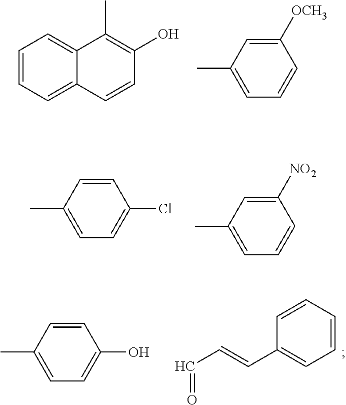
and pharmaceutically acceptable salts thereof.

2. The compound of claim 1, wherein said pharmaceutically acceptable salts consist of base addition salts.

3. The compound of claim 1 wherein the salt is selected from an alkali metal salts of Li, Na or K and an alkaline earth metal salt of Ca or Mg.

4. The compound of claim 1 wherein the salt selected is a salt of an organic base and an ammonium or a substituted ammonium salt.

5. The compound of claim 1 wherein the salt is selected from a sulphate, a nitrate, a phosphate, a perchlorate, a borate, a hydro halide, an acetate, a tartrate, a malate, a citrate, a succinate, a palmoate, a methanesulfonates, a benzoate, an ascorbate, a glycerophosphate and a ketoglutarate salt.

6. The compound of claim 1 wherein R2 is selected from the group consisting of Aryl, substituted aryl groups.

7. A method of treatment of a disease comprising administering to a subject suffering from a cancer, the compound of claim 1.

8. The method of claim 7 wherein the cancer is selected from: breast cancer, lung cancer, skin cancer, prostate cancer, and colorectal cancer.
